(19) United States
(12) Patent Application Publication
(10) Pub. No.: US 2021/0353655 A1
(43) Pub. Date: Nov. 18, 2021

(54) PLANT-BASED PHYTONUTRIENT COMPOSITION FOR REGULATION OF SIGNAL TRANSDUCTION CASCADES IN THE BODY AND RELATED METHODS

(71) Applicant: Brilliant Lab LLC, American Fork, UT (US)

(72) Inventor: Daniel A. Gubler, Ph.D., Orem, UT (US)

(73) Assignee: Brilliant Lab LLC, American Fork, UT (US)

(21) Appl. No.: 17/230,436

(22) Filed: Apr. 14, 2021

Related U.S. Application Data
(60) Provisional application No. 63/025,425, filed on May 15, 2020.
(60) Provisional application No. 63/122,355, filed on Dec. 7, 2020.

(51) Int. Cl. A61K 31/704; A61K 31/05; A61K 31/4375; A61K 31/353; A61K 36/288; A61K 31/12; A61K 36/074; A61K 31/7016; A61K 31/366; A61K 9/00; A61K 9/48; A61K 9/20; A61P 5/24

(52) U.S. Cl. CPC A61K 31/704; A61K 31/05; A61K 31/4375; A61K 31/353; A61K 36/288; A61K 31/12; A61P 5/24; A61K 31/7016; A61K 31/366; A61K 9/0053; A61K 9/4825; A61K 9/20; A61K 36/074

(57) ABSTRACT

Described, among other things, are dosage form for administration to a subject, the dosage forms having at least eight phytonutrients selected from resveratrol, berberine, epigallocatechin gallate, quercetin, dandelion root extract powder, curcuminoids, burdock root powder, Reishi mushroom extract powder, African mango seed extract, rosavins, ginkgolides, ellagic acid, Ginsengosides, and gingerols, The dosage forms can serve as biosignaling phytonutrient multi-supplement compositions capable of promoting a subject's health through the maintenance of at least one signal transduction cascade. In certain embodiments, the dosage form is administered to the, e.g., human female subject in order to reduce the symptoms of premenstrual syndrome in the subject.

### CROSS-REFERENCE TO RELATED APPLICATIONS

This application claims the benefit under 35 U.S.C. § 119(e) of U.S. Provisional Patent Application Ser. No. 63/025,425, filed May 15, 2020 and of U.S. Provisional Patent Application Ser. No. 63/122,355, filed Dec. 7, 2020, the disclosure of the entirety of each of which is hereby incorporated herein by this reference.

### TECHNICAL FIELD

The application relates generally to nutritional supplements and associated methods of making and using them. More particularly, the application relates to a supplement containing multiple, select phytonutrients useful for, among other things, promoting a subject's health through the upkeep of signal transduction cascades in the subject.

### BACKGROUND

In the human body, millions of chemical reactions are performed every second. The sum total of the chemical reactions that occur in an organism constitutes the metabolism of the organism. These chemicals reactions are originated and orchestrated by many different biosignaling pathways in the body known as signal transduction cascades (“STCs”).

STCs are the means by which the body sends signals inside the cell, between cells, and throughout the entire body. STCs are the genesis of mammalian (e.g., human) health and are triggered by stimuli-like small molecules, light, heat, and mechanical force. There are generally conserved STCs throughout the body, and there are STCs specific to different organs and systems in the body. Small molecules, also called ligands, are one of the most commonly conserved triggers for STCs.

A simple way to think about STCs is likening them to a line of dominoes that cause a cascade of falling dominoes, which trigger a chain reaction that accomplishes the metabolic goal. Small molecules, proteins, and other stimuli are akin to dominoes in biological systems that pass signal on to the next partner in the chain. These biosignals eventually reach the nucleus or other organelles that turn on genes, proteins, or other systems controlling essential metabolic reactions. Lifestyle factors like poor diet, lack of sleep, lack of exercise, stress, anxiety, pollution, smoking, and depression can cause these biosignaling reactions to be reduced in frequency or even stop all-together. Using the domino analogy, these factors remove certain small molecules, or dominoes, out of the cascade that, in turn, causes reduced function of the STCs.

### BRIEF SUMMARY

Described are phytonutrient-containing supplement compositions useful to promote health of the generally conserved signal transduction cascades (“STCs”) in the body. The described phytonutrient compositions are useful in maintaining metabolic health of a subject (e.g., a mammal such as a human). The supplements help to address the “preventative well-care need state” of the subject, which is not currently addressed by, e.g., the daily administration of multivitamins. In certain embodiments, the described phytonutrient compositions are useful in reducing the symptoms of premenstrual syndrome (“PMS”) in a female subject.

Also described is the use of specific, selected phytonutrients with biosignaling capabilities that are administered to the subject for use in maintaining the robustness of the generally conserved STCs in a subject's body.

Further described is a composition of a combination of multiple, selected phytonutrients, which combination is able to modulate (e.g., upregulate) different signal transduction pathways.

Particularly described herein are dosage forms for administration to a subject, each dosage form including at least eight phytonutrients selected from the group consisting of resveratrol, berberine, epigallocatechin gallate, quercetin, dandelion root extract powder, curcuminoids, burdock root powder, Reishi mushroom extract powder, African mango seed extract, rosavins, ginkgolides, ellagic acid, Ginsengosides, and Gingerols. In certain embodiments, the selected phytonutrients may be combined with one or more other agents, both pharmacologically active and inactive (e.g., at least one pharmaceutically acceptable excipient). Typically, the dosage form will be in a form selected from the group consisting of a softgel, capsule, tablet, gel, powder, gummy, liquid, effervescent, bar, topical patch, serum, lotion, and cream.

In certain embodiments, the selected phytonutrients may be administered in two different dosage forms, each containing fewer than eight of the selected phytonutrients, but intended to be co-administered to the subject in need thereof

Thus, also described is a method of maintaining at least one generally conserved signal transduction cascade (“STC”) in a subject, the method comprising: administering a combination of at least eight selected phytonutrients to the subject, wherein the at least eight phytonutrients are selected are from the group consisting of resveratrol, berberine, epigallocatechin gallate, quercetin, dandelion root extract powder, curcuminoids, burdock root powder, Reishi mushroom extract powder, African mango seed extract, rosavins, ginkgolides, ellagic acid, Ginsengosides, and gingerols,

Typically, the dosage form contains sufficient amounts of the selected at least eight phytonutrients to maintain health of at least one generally conserved signal transduction cascades (“STCs”) in the subject when the subject ingests at least one of the dosage forms on a daily basis. For example, the dosage form contains the selected at least eight phytonutrients present in the following amounts (when they are present): from about 50 to about 250 milligrams of resveratrol, from about 100 to about 500 milligrams of berberine, from about 100 to about 400 milligrams of epigallocatechin gallate, from about 20 to about 100 milligrams of quercetin, from about 100 to about 300 milligrams of dandelion root extract powder, from about 20 to about 80 milligrams of curcuminoids, from about 75 to about 300 milligrams of burdock root powder, from about 50 to about 200 milligrams of Reishi mushroom extract powder, from about 75 to about 450 milligrams of African mango seed extract, from about 2 to about 10 milligrams of Rosavins, from about 20 to about 60 milligrams of Ginkgolides, from about 50 to about 110 milligrams ellagic acid, from about 5 to about 25 milligrams of Ginsengosides, and from about 2 to about 20 milligrams of Gingerols.

Such dosage forms can be made by admixing the selected phytonutrients and associating them together into or with the dosage form.

In certain embodiments, the dosage form has phytonutrients selected from the group consisting of resveratrol, berberine, epigallocatechin gallate, quercetin, curcuminoids, burdock root powder, rosavins, ginkgolides, and ellagic acid.

The dosage form is preferably orally administered to the subject, but other administration modalities are contemplated. When administered orally, the subject ingests the selected at least eight phytonutrients, when present, in the following amounts on a daily basis: from about 50 to about 250 milligrams of resveratrol, from about 100 to about 500 milligrams of berberine, from about 100 to about 400 milligrams of epigallocatechin gallate, from about 20 to about 100 milligrams of quercetin, from about 100 to about 300 milligrams of dandelion root extract powder, from about 20 to about 80 milligrams of curcuminoids, from about 75 to about 300 milligrams of burdock root powder, from about 50 to about 200 milligrams of Reishi mushroom extract powder, from about 75 to about 450 milligrams of African mango seed extract, from about 2 to about 10 milligrams of Rosavins, from about 20 to about 60 milligrams of Ginkgolides, from about 50 to about 110 milligrams ellagic acid, from about 5 to about 25 milligrams of Ginsengosides, and from about 2 to about 20 milligrams of Gingerols.

In certain embodiments, the dosage form may be administered to the, e.g., human subject in order to reduce interference with an STC caused by at least one environmental factor to which the subject is subjected (pollution, smoking, etc.). Such STCs include TGF-Beta/Smad2, Bcl2/BAX/P53, TGF-Beta/JNK, iNOS, NADPH Oxidase 4, WNT/Beta-Catenin, STAT3, MAPK/Capsase-3, TRAIL Lectin-like ox-LDL-receptor-1, JAK-STAT, IRF3, Mst1-JNK, PI3K/AKT/mTOR, JNK, TLR4/MyD88/NF-κB, Dipeptidyl Peptidase-4, IRS1-GLUT4, ERK ½, Skp2-p27, PI3K/Akt, AP-1, Hedgehog, Adiponectin-AMPK. Akt/GSK-3-beta, RAF, ATM/ATR, MAPK/Erk, IKK/NF-κB, p38/ERK, TNF-α, Protein Tyrosine Phosphatase 1B, JNK/ERK VEGFR1 & VEGFR2, Ca 2+ levels, FOX, IRS/MAPK, NF-κB, IL-6 & IL-8, NF-κB/MAPK, COX-2, IL-8, and any combination thereof.

In certain embodiments, the dosage form is administered to the, e.g., human female subject for use in reducing the symptoms of premenstrual syndrome (“PMS”) in the subject.

Preferably, the STC(s) comprise(s) at least one selected from the group consisting of NF-κB, MAPK, COX-2, and JNK/ERK. In certain embodiments, the STCs maintained are NF-κB and MAPK, which STCs react synergistically with each other. In certain embodiments, the STCs are COX-2 and JNK/ERK, which STCs react synergistically with each other.

### BRIEF DESCRIPTION OF THE FIGURES

FIG. 1 are graphs depicting the viability of cells treated with the composition of EXAMPLE IV for hTERT-HPNE (Top) and Panc-1 (Bottom) cells.

FIG. 2 are graphs depicting the gene expression of cyclin D1 in hTERT-HPNE (Top) and Panc-1 (Bottom).

FIG. 3 are graphs depicting the gene expression of Cox-2 in hTERT-HPNE (Top) and Panc-1 (Bottom).

FIG. 4 are graphs depicting the gene expression of c-Myc in hTERT-HPNE (Top) and Panc-1 (Bottom).

FIG. 5 are graphs depicting the gene expression of PI3K in hTERT-HPNE (Top) and Panc-1 (Bottom).

FIG. 6 are graphs depicting the gene expression of PPARγ in hTERT-HPNE (Top) and Panc-1 (Bottom).

FIG. 7 are graphs depicting the gene expression of PPARγ in hTERT-HPNE (Top) and Panc-1 (Bottom) with the 16-hour time point not shown.

FIG. 8 are graphs depicting the relative levels of phosphoproteins HSP27, JNK, MEK1, and P38 in response to treatment with a composition as described herein.

FIG. 8A is a graph depicting the relative levels of phosphoproteins P58 in response to treatment with the composition.

FIG. 9 is a graph describing the relative levels of phosphoprotein MSK2 in response to treatment with the composition.

FIG. 10 are graphs depicting the relative levels of phosphoproteins CREB, MKK6 and MKK3 in response to treatment with the composition.

FIG. 11 are graphs depicting the relative levels of phosphoproteins RSK1, RSK2 and ERK½ in response to treatment with the composition.

FIG. 12 are graphs depicting the relative levels of phosphoproteins AKT, AMPKa, BAD, and GSk3a in response to treatment with the composition.

FIG. 12A are graphs depicting the relative levels of phosphoproteins P70S6k, mTOR, P27, and PDK1 in response to treatment with the composition.

FIG. 12B are graphs depicting the relative levels of phosphoproteins PTEN, Raf-1, and RPS6, in response to treatment with the composition.

FIG. 13 is a graph depicting the relative levels of phosphoprotein 4E-BP-1, in response to treatment with the composition.

FIG. 14 are graphs depicting relative levels of phosphoproteins JAK1, SHP2, STAT3, and STATS in response to treatment with the composition.

FIG. 14A is a graph depicting the relative levels of phosphoprotein TYK2, in response to treatment with the composition.

FIG. 15 are graphs depicting the relative levels of phosphoproteins Stat1 and Stat2, in response to treatment with the composition.

FIG. 16 are graphs depicting relative levels of phosphoproteins eIF2a, HDAC2, HDAC4, and IkBa in response to treatment with the composition.

FIG. 16A are graphs depicting relative levels of phosphoproteins MSK1, TBK1 and ZAP70, in response to treatment with the composition.

FIG. 17 are graphs depicting relative levels of phosphoproteins ATM and TAK1, in response to treatment with the composition.

FIG. 18 are graphs depicting relative levels of phosphoproteins ATF2, Smad4 and Smad5, in response to treatment with the composition.

FIG. 19 is a graph depicting relative levels of phosphoprotein c-Jun in response to treatment with the composition.

FIG. 20 is a graph depicting elative levels of phosphoprotein Smad-1 in response to treatment with the composition.

FIG. 21 is a graph showing that a composition as described herein reduces salivary cortisol levels: Saliva samples were collected on Days 0, 7, and 21 from 15 participants who took the composition for three weeks. Cortisol levels were then analyzed. Mean cortisol levels were used to compare between each time point and error bars represent the standard error of the mean (“SEM”).

FIG. 22 is a graph showing that a composition as described herein reduces salivary levels of TNF-α: Saliva samples were collected on Days 0, 7, and 21 from 15 participants who took the composition for three weeks. TNF-α levels were then analyzed. Mean TNF-α levels were used to compare between each time point and error bars represent the SEM.

FIG. 23 is a graph showing that a composition as described herein reduces salivary levels of IL-8: Saliva samples were collected on Days 0, 7, and 21 from fifteen participants who took the composition of Example IV for three weeks. IL-8 levels were then analyzed. Mean IL-8 level was used to compare between each time point and error bars represent the SEM.

### DETAILED DESCRIPTION

Described is a biosignaling composition comprising selected phytonutrients that can be included in a multivitamin-nutritional supplement dosage form to support health via the everyday preventative well-care approach espoused by polypharmacology. This composition may be formulated and administered to help maintain human health more effectively than the classic daily therapy of daily multivitamins comprising vitamins and minerals.

For purposes of this disclosure, major chemical derivatives of plants fall into three categories, i.e., 1. Macronutrients (e.g., fat, protein, and carbohydrates), 2. Micronutrients (e.g., vitamins and minerals), and 3. Phytonutrients (e.g., organic compounds like polyphenols, sulforaphanes, amines, amides, lactones, ketones, etc.).

Phytonutrients are the “medicinal” component and are commonly a starting point in the drug discovery process.

Phytonutrients described herein have generally been extracted (e.g., from raw plant/fungal material) and/or standardized to the respective compounds for assimilation into the dosage form(s). Such processing increases reproducibility and quality, and reduces the bulk of the selected phytonutrients and allows for smaller, easier to swallow dosage forms.

Although the phytonutrients are generally readily commercially available, processes for extracting phytonutrients are known in the art. See, e.g., Wang et al. “A simple method for the isolation and purification of resveratrol from Polygonum cuspidatum,” Journal of Pharmaceutical Analysis, 3(4):241-247 (2013), the contents of which are incorporated by reference.

In certain embodiments, the dosage form is utilized or administered together with other organic or inorganic compounds in order to support the overall need state addressed by the product (e.g., energy, cognitive health, immune health, joint health, etc.).

In certain embodiments, the dosage form is administered daily to a subject due to its ability to promote health of the general chemical reactions in the body that need to occur for proper function.

Examples of biosignaling phytonutrients that are useful herein include resveratrol, berberine, epigallocatechin-3-gallate, quercetin, curcumin, rosavin, Ginkgolide A, ellagic acid, ginsengoside, and gingerol.

Resveratrol is a phytonutrient that is readily commercially available. It regulates, among other things, the following STC(s): NF-κB/MAPK, and COX-2. It has anti-inflammatory properties. As used herein, a typical daily dosage form (e.g., for use with an adult human subject). As used herein, a typical daily dosage form (e.g., for use with an adult human subject) contains from about (plus or minus 5% by weight) 50 to about 250 milligrams of resveratrol. The amount can be adjusted for divided doses of the dosage form.

Berberine is a phytonutrient that is readily commercially available. It regulates, among other things, the following STC(s): NF-κB, Set9, AMP, 3T3-L1, PPAR, and LDLR. It regulates cholesterol biosynthesis and glucose uptake, and is anti-inflammatory. As used herein, a typical daily dosage form (e.g., for use with an adult human subject) contains from about (plus or minus 5% by weight) 100 to about 500 milligrams of berberine. The amount can be adjusted for divided doses of the dosage form.

Epigallocatechin gallate (“EGCG”) is a phytonutrient that is readily commercially available. It regulates, among other things, the following STC(s): FOX, IRS/MAPK, TNF-α, IL-8, and PGE2. It regulates antioxidant cascades and energy expenditure, and has anti-inflammatory properties. As used herein, a typical daily dosage form (e.g., for use with an adult human subject) contains from about (plus or minus 5% by weight) 100 to about 400 milligrams of epigallocatechin gallate. The amount can be adjusted for divided doses of the dosage form. Green tea extract may be standardized to 60% EGCG.

Quercetin is a phytonutrient that is readily commercially available. It regulates, among other things, NF-κB/MAPK. It is anti-inflammatory, and regulates the cell cycle. As used herein, a typical daily dosage form (e.g., for use with an adult human subject) contains from about (plus or minus 5% by weight) 20 to about 100 milligrams of quercetin. The amount can be adjusted for divided doses of the dosage form. Quercetin dihydrate may be standardized to quercetin.

Dandelion root extract powder is a phytonutrient that is readily commercially available. It regulates, among other things, Ca 2+ levels. It affects intercellular signaling, cell transport, and bone health. As used herein, a typical daily dosage form (e.g., for use with an adult human subject) contains from about (plus or minus 5% by weight) 100 to about 300 milligrams of dandelion root extract powder. The amount can be adjusted for divided doses of the dosage form.

Curcuminoids is a phytonutrient composition that is readily commercially available. It regulates, among other things, the following STC(s): ERK ½, TNF-α, IL-8, and JNK/ERK. It is anti-inflammatory, and regulates cell growth and cell death. As used herein, a typical daily dosage form (e.g., for use with an adult human subject) contains from about (plus or minus 5% by weight) 20 to about 80 milligrams of curcuminoids. The amount can be adjusted for divided doses of the dosage form. Curcuminoids may be standardized to 95% curcuminoids.

Burdock root powder is a phytonutrient that is readily commercially available. It regulates, among other things, STAT3. It has effects on immune function and cell growth, and is anti-inflammatory. As used herein, a typical daily dosage form (e.g., for use with an adult human subject) contains from about (plus or minus 5% by weight) 75 to about 300 milligrams of burdock root powder. The amount can be adjusted for divided doses of the dosage form.

Reishi mushroom extract powder is a phytonutrient that is readily commercially available. It regulates, among other things, Protein Tyrosine Phosphatase 1B. It affects insulin signaling, and has benefits in weight loss, fat burning, and glucose control. As used herein, a typical daily dosage form (e.g., for use with an adult human subject) contains from about (plus or minus 5% by weight) 50 to about 200 milligrams of Reishi mushroom extract powder. The amount can be adjusted for divided doses of the dosage form. Reishi mushroom extract may be standardized to 30% polysaccharides.

African mango seed extract is a phytonutrient that is readily commercially available. It regulates, among other things, PPAR, and affects metabolic health, fat burning, and cell differentiation. As used herein, a typical daily dosage form (e.g., for use with an adult human subject) contains from about (plus or minus 5% by weight) 75 to about 450 milligrams of African mango seed extract. The amount can be adjusted for divided doses of the dosage form.

Rosavins is a phytonutrient (e.g., from Rhodiola rosea) that is readily commercially available. It regulates, among other things, the following STC(s): TNF-α, NF-κB, IL-6, JAK/STAT, and IL-8. It has anti-inflammatory and antioxidant properties. As used herein, a typical daily dosage form (e.g., for use with an adult human subject) contains from about (plus or minus 5% by weight) 2 to about 10 milligrams of Rosavins. The amount can be adjusted for divided doses of the dosage form. R. rosea may be standardized to 3% Rosavins, 1% salidrosides.

Ginkgolides is a phytonutrient that is readily commercially available. It regulates, among other things, Lectin-like ox-LDL-receptor-1, and affects cholesterol biosynthesis and control. As used herein, a typical daily dosage form (e.g., for use with an adult human subject) contains from about (plus or minus 5% by weight) 20 to about 60 milligrams of Ginkgolides. The amount can be adjusted for divided doses of the dosage form. Ginkgo biloba 24/6 may be standardized to 24% flavone glycosides, 6% terpene lactones.

Ellagic acid is a phytonutrient (e.g., from pomegranate) that is readily commercially available. It regulates, among other things, the following STC(s): NF-κB/MAPK Anti-inflammatory, cell cycle, and Wnt signaling pathway. As used herein, a typical daily dosage form (e.g., for use with an adult human subject) contains from about (plus or minus 5% by weight) 50 to about 110 milligrams ellagic acid. The amount can be adjusted for divided doses of the dosage form. Pomegranate extract powder 5:1 may be standardized to 40% ellagic acid.

Ginsengosides are phytonutrients that are readily commercially available. They regulate, among other things, the following STC(s): NF-κB, AP1, and ERK. It affects cell growth, cell death, and is anti-inflammatory, and assists in wound healing. As used herein, a typical daily dosage form (e.g., for use with an adult human subject) contains from about (plus or minus 5% by weight) 5 to about 25 milligrams of Ginsengosides. The amount can be adjusted for divided doses of the dosage form.

Gingerols are phytonutrients that are readily commercially available. Gingerols regulate, among other things, the following STC(s): NF-κB, AP1, and beta-catenin. Gingerols affect cell growth, cell death, and are anti-inflammatory. As used herein, a typical daily dosage form (e.g., for use with an adult human subject) contains from about (plus or minus 5% by weight) 2 to about 20 milligrams of Gingerols. The amount can be adjusted for divided doses of the dosage form.

Signal Transduction Cascade Biosignaling Pathways relevant to this disclosure include TGF-Beta/Smad2, Bcl2/BAX/P53, TGF-Beta/JNK, iNOS, NADPH Oxidase 4, WNT/Beta-Catenin, STAT3, MAPK/Capsase-3, TRAIL Lectin-like ox-LDL-receptor-1, JAK-STAT, IRF3, Mst1-JNK, PI3K/AKT/mTOR, JNK, TLR4/MyD88/NF-κB, Dipeptidyl Peptidase-4, IRS1-GLUT4, ERK ½, Skp2-p27, PI3K/Akt, AP-1, Hedgehog, Adiponectin-AMPK. Akt/GSK-3-beta, RAF, ATM/ATR, MAPK/Erk, IKK/NF-κB, p38/ERK, TNF-α, Protein Tyrosine Phosphatase 1B, JNK/ERK VEGFR1 & VEGFR2, Ca 2+ levels, FOX, IRS/MAPK, NF-κB, IL-6 & IL-8, NF-κB/MAPK, COX-2, and IL-8.

While not intending to be bound by theory, the described components of the combinations work by different mechanisms to regulate different STCs, e.g., as described herein. Thus, using select combinations of the different components in a single dosage form, some components having a different mechanism and spectrum of activity (as described herein) than others acts to regulate the subject's STCs in a manner greater and differently than the individual components would, allowing for outcomes that the individual components could not have, and enlarging their range of utility.

Once being apprised of the instant disclosure, a person of ordinary skill in the art will be readily able to make or prepare the dosage forms using Galenical techniques. Preferably, a finished product delivery forms is selected from the group consisting of a softgel, capsule, tablet, gel, powder, gummy, liquid, effervescent, bar, topical patch, serum, lotion, and cream.

The invention is further described with the aid of the following illustrative EXAMPLES.

### EXAMPLE I

The following phytonutrients are thoroughly admixed as close to uniform consistency as possible:

| | | |
|---|---|---|
| Resveratrol | 55 | grams |
| Berberine | 150 | grams |
| Epigallocatechin gallate | 125 | grams |
| Quercetin | 30 | grams |
| Dandelion root extract powder | 100 | grams |
| Curcuminoids | 25 | grams |
| Burdock root powder | 90 | grams |
| Reishi mushroom extract powder | 62.5 | grams |
| African mango seed extract | 130 | grams |
| Rosavins | 3 | grams |
| Ginkgolides | 20 | grams |
| Ellagic acid | 35 | grams |
| Ginsengosides | 7.5 | grams |
| Gingerols | 5 | grams |

The resulting admixture is divided into 1,000 equal portions each placed into one of 1,000 appropriately-sized hard (or vegan) gelatin capsules.

### EXAMPLE II

Capsules of EXAMPLE I are administered in a dosing regimen of one capsule twice daily (e.g., with breakfast and dinner) to a subject suffering from inflammation. The treatment regimen is continued for 13 weeks. The described phytonutrient-based biosignaling supplement supports the subject's health via, for example, a preventative well-care approach. Furthermore, inflammation is reduced in the subject.

### EXAMPLE III

The following phytonutrients are thoroughly admixed:

| | | |
|---|---|---|
| Resveratrol | 1.5 | grams |
| Berberine | 3 | grams |
| Epigallocatechin gallate | 2.5 | grams |
| Quercetin | 600 | milligrams |
| Curcuminoids | 500 | milligrams |
| Burdock root powder | 1,800 | milligrams |
| Rosavins | 60 | milligrams |
| Ginkgolides | 400 | milligrams |
| Ellagic acid | 700 | milligrams |

The resulting admixture is divided into ten equal portions and each portion is placed into one of ten appropriately-sized hard gelatine (or vegan) capsules.

### EXAMPLE IV

The following phytonutrients are thoroughly admixed:

| | |
|---|---|
| Pomegranate Extract Powder 5:1 | 6.96 g |
| Berberine | 6.96 g |
| Green Tea Extract | 5.82 g |
| Dandelion Root Extract 4:1 | 5.22 g |
| Burdock Root Extract | 5.22 g |
| Reishi Mushroom Extract | 3.48 g |
| Resveratrol | 3.48 g |
| African Mango Seed Extract 4:1 | 3.48 g |
| Rhodiola rosea | 5.22 g |
| Ginkgo biloba 24/6 | 5.22 g |
| Quercetin Dihydrate | 1.74 g |
| Curcuminoids | 1.74 g |

The resulting admixture is divided into sixty equal portions and tabletted into sixty equivalent tablets. The tablets are intended for twice a day administration or to be taken two at a time.

### EXAMPLE V

Two tablets of EXAMPLE IV were administered daily to each of a group of women. 14 of the women taking the composition noticed a significant relief in pain related to PMS that was as efficacious to them as taking ibuprofen. Menstrual cramping and bleeding were reduced in severity by 50%. All of these women also reported that the mood depressions related to PMS were essentially gone.

### EXAMPLE VI

This Example evaluates the effect(s) of the ingredients of the composition of EXAMPLE IV on the gene expression levels of PI3K, Cox-2 (PTGS2), cyclin D1 (surrogate for STAT3), c-myc and PPARg (ml PPARGC1) by qRT-PCR. Additionally, the effect of the composition of EXAMPLE IV on the level of phosphorylated proteins in five human cell signaling pathways including the MAPK, AKT, JAK/STAT, NFkb and TGFb pathways was evaluated using a Human Phosphorylation Pathway Profiling Array (Ray Biotech, Peachtree Corners, Georgia, US).

Materials and Methods. Test compounds. Capsules containing the composition of EXAMPLE IV was provided by Brilliant Science LLC as capsules rather than as tablets.

Stock solutions. The composition of EXAMPLE IV powder was dissolved or suspended in DMSO, to a concentration of 10 and 100 mg/mL. Further working stocks were made in RPMI media.

Cell lines and culture. hTERT-HPNE is an immortalized normal human pancreatic cell line (ATCC, CRL-4023). Panc-1 is a pancreatic cancer cell line isolated from a 56 year old male Caucasian (ATCC, CRL-1469). hTERT-HPNE cells were cultured in 75% DMEM/25% M3 Base/5% fetal bovine serum/10 ng/ml human recombinant EGF/5.5 mM D-glucose. Panc-1 cells were cultured in RPMI 1640 culture medium with L-glutamine (Thermo-Fisher Scientific, Waltham Mass., Catalog #11875-093), supplemented with 10% fetal bovine serum. Viability of cells was assessed by daily monitoring for fungal contamination, turbidity, pH shift (phenol red), correct morphology, expected number of cells in mitosis (rounded-up). Furthermore, cells were assessed for trypan blue exclusion when passaging to verify that there was not an unexpected percentage of non-viable cells (<2% non-viable cells).

Cell viability/ATP content assays. The Cell-Titer-Glo Luminescent Cell viability assay (Promega) is a method to determine the number of viable cells in culture based on quantitation of the ATP present, as an indicator of metabolically active cells. Cells were seeded at a density of from 125-2000 cells/well in 384-well white-walled Cultur-Plate-384 cell culture plates (Perkin-Elmer, Waltham Mass., Catalog #6007680). Dilutions of The composition of EXAMPLE IV were added to wells of the plate in quadruplicate. Cells were allowed to grow in the presence of The composition of EXAMPLE IV agent for 24 hours for dose-course studies, at which point Cell-Titer-Glo (Promega Corp., Madison Wis., Catalog #G7571) reagent was added at a volume equal to the cell culture medium in the plate (50 μL+50 μL), according to manufacturer's instructions. Luminescence was read on an Envision 2104 multilabel reader (Perkin-Elmer, Waltham Wis.). A 700 nm filter was used to reduce background signal greater than 700 nM (bioluminescence of luciferase is 562 nm).

Gene expression assay. hTERT-HPNE cells were seeded in T75 flasks at 1.5 million cells per flask in hTERT-HPNE medium. Panc-1 cells were seeded at 3 million cells per flask in RPMI medium. Flasks were prepared in duplicate for an untreated control and 2-, 6- and 16-hour treatment time points. After 24 hours of incubation, cells were treated with The composition of EXAMPLE IV at 100 ug/mL and at the indicated times the cells were harvested, lysed with lysis buffer and RNA isolations were conducted per manufacturer's recommendations using the Quick-RNA MiniPrep kit (Zymo Research, Cat. No. 11-327). RNA was eluted with RNase free water and quantified by spectrophotometer. Reverse transcription reactions were performed using 1 μg of RNA using the Vilo Superscript system (Thermo, Cat. No. 11756500). A ten-fold dilution was made of each cDNA in PCR-grade water, and 8 μL of this solution was carried forward into qRT-PCR.

Reactions were carried out in 20 μL total volume, made up as follows: 8 μL cDNA, 1 μL gene-of-interest primer (FAM label), 1 μL hACT primer (VIC label), 10 μL Taqman Fast Advanced Master Mix (Thermo Fisher Scientific, Catalog #4444963). Reactions were run on an Applied Biosystems 7500 Fast Real-Time PCR Instrument (Thermo Fisher Scientific) under the following conditions: 50° C.—2 minutes, 95° C.—20 seconds, 40 cycles of (95° C.—3 seconds, 60° C.—30 seconds). Threshold cycle (CT) was determined by the instrument software. Differences in threshold cycle between the gene of interest and hACT (ACT) were determined for each sample and used to determine fold induction of each gene of interest, compared to untreated controls.
PCR primers were purchased from Thermo Fisher Scientific as follows:

PIK3CA (PI3K): Hs00907957_m1 (FAM labeled)

PTGS2 (Cox-2): Hs00153133_m1 (FAM labeled)

CCND1 (cyclin D1): Hs00765553_m1 (FAM labeled)

MYC (c-myc): Hs00153408_m1 (FAM labeled)

PPARGC1A (PPAR g): Hs01016719_m1 (FAM labeled)

hACTB control (human b actin): Hs99999903_m1 (VIC labeled)

Human Phosphorylation Pathway Profiling Array assay. hTERT-HPNE and Panc-1 cells were seeded into two T182 flasks each, a control and a treatment flask. When the cells were 30% confluent, one flask from each cell line was administered the composition of EXAMPLE IV at 100 ug per mL. The cells were incubated for 2-hours and the cells were harvested from all four flasks. Cell lysates were prepared from the harvested cells as per the manufacturer's instructions (Ray Biotech, Human Phosphorylation Pathway Profiling Array C55 kit, Catalog # AAH-PPP-1-4). The quantities of protein in each cell lysate was determined using a BCA Protein Assay kit (Pierce, Catalog #23227). The Human Phosphorylation Pathway Profiling Array assay was performed as per the manufacturer's instructions. The kit comes with 5 separate arrays, one for each of the 5 cell signaling pathways examined, and 4 sets of the five arrays to analyze an untreated and the composition of EXAMPLE IV-treated sample of each of the two cell lines examined. Briefly, the filters were blocked using blocking buffer at room temperature for 30 minutes. The blocking buffer was removed, and the arrays were incubated with the cell lysates, untreated and BT-treated for both Panc-1 and hTERT-HPNE cell lines, for 3 hours to overnight. The arrays were washed 3 times with Washing Buffer 1 for 5 minutes at room temperature with shaking. The arrays were washed 2 times with Washing Buffer 2 for 5 minutes at room temperature with shaking. The Washing Buffer was aspirated, and individual arrays were incubated with one of five Detection Antibody Cocktails at 4 degrees C. overnight. The arrays were washed 3 times with Washing Buffer 1 for 5 minutes at room temperature with shaking. The arrays were washed 2 times with Washing Buffer 2 for 5 minutes at room temperature with shaking. The Washing Buffer was aspirated, and the arrays were incubated with HRP-Anti-Rabbit IgG at room temperature for 2 hours. The arrays were washed 3 times with Washing Buffer 1 for 5 minutes at room temperature with shaking. The arrays were washed 2 times with Washing Buffer 2 for 5 minutes at room temperature with shaking. The Washing Buffer was aspirated, and the Detection Buffer mix was applied to the arrays, the arrays were covered with a plastic film, and detection was performed using an iBright 1500 western blot imaging system (ThermoFisher).

Data analysis was performed as follows. The raw data from the iBright imager was extracted, the background was subtracted and the data was normalized to the Positive Control signal spots on the arrays. Background for subtraction is based on the values for the Negative Control Spots (NEG). The amount of Detection antibody printed for each Positive Control Spot is consistent from array to array. As such, the intensity of these Positive Control signals can be used to normalize signal responses for comparison of results across multiple arrays, much like housekeeping genes and proteins are used to normalize results of PCR gels and Western Blots, respectively.

To normalize the array data, one array was defined as the “Reference Array”, the untreated Panc-1 sample was used for this purpose, to which the other arrays were normalized to. The fold expression between samples was calculated using the following formula:

X(Ny)=X(y)*P1/P(y)

### Where:

P1=mean signal density of Positive Control spots on the reference array

P(y)=mean signal density of Positive Control spots on Array “y”

X(y)=mean signal density for spot “X” on Array “y”

X(Ny)=normalized signal intensity for spot “X” on Array “y”

Every protein of interest is represented by two spots on the arrays and the positive and negative controls are based on up to 6 spots each. The standard deviation and relative error are calculated based on the sample replicates.

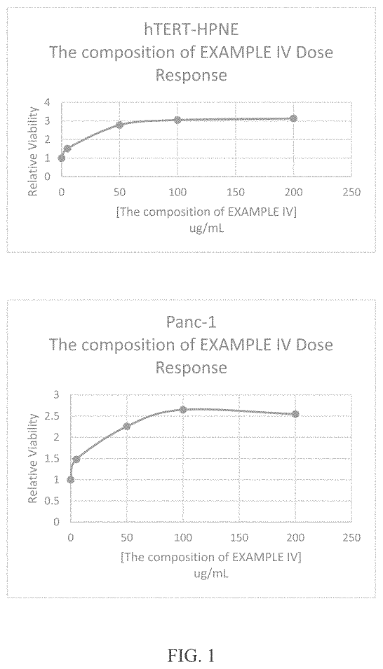

Results and Discussion: Viability/dose finding assay. In order to determine the proper dosage level for the composition of EXAMPLE IV, a concentration gradient was set up in 384-well plates, and viability was tested using the Cell Titer-Glo assay (Promega). The assay was carried out as described in the materials and methods section, and resulting dose-dependent curves are shown in FIG. 1.

Based on the results, the composition of EXAMPLE IV was well tolerated by both cell lines. In fact, contrary to most studies that we have performed with other materials, the composition of EXAMPLE IV appears to promote growth for both the normal (hTERT-HPNE) and cancerous (Panc-1) pancreatic cell lines. Based on the excellent safety profile seen from the cell viability data, it was determined that the qRT-PCR and Human Phosphorylation Pathway Profiling Array assays would be conducted at 100 μg/mL The composition of EXAMPLE IV, to maximize the expected responses.

qRT-PCR gene expression. Taqman probe gene expression assays were carried out at 100μg/mL to determine the effects of The composition of EXAMPLE IV on the expression of the genes as shown in Table 1 (Gene expression targets):

TABLE 1

| | Gene | TaqMan |
|---|---|---|
| Gene Target | Symbol | Assay ID |
| phosphatidylinositol-4,5-bisphosphate | PIK3CA | Hs00907957_m1 |
| 3-kinase catalytic subunit alpha | | |
| (PI3K) | | |
| prostaglandin-endoperoxide synthase | PTGS2 | Hs00153133_m1 |
| 2 (Cox-2) | | |
| cyclin D1 | CCND1 | Hs00765553_m1 |
| v-myc avian myelocytomatosis viral | MYC | Hs00153408_m1 |
| oncogene homolog (c-Myc) | | |
| PPARG coactivator 1 alpha (PPAR g) | PPARGC1A | Hs01016719_m1 |
| b actin (Reference) | ACTB | Hs99999903 |

hTERT-HPNE and PANC-1 cells, untreated and the composition of EXAMPLE IV-treated (100 ug/mL) were evaluated for mRNA expression levels for the five genes of interest and the b-actin reference (Table 1). For the composition of EXAMPLE IV treatment, 2-, 6- and 16-hour time points were examined. For all conditions, two biological replicates were analyzed, and duplicate technical replicates of these were examined by qRT-PCR.

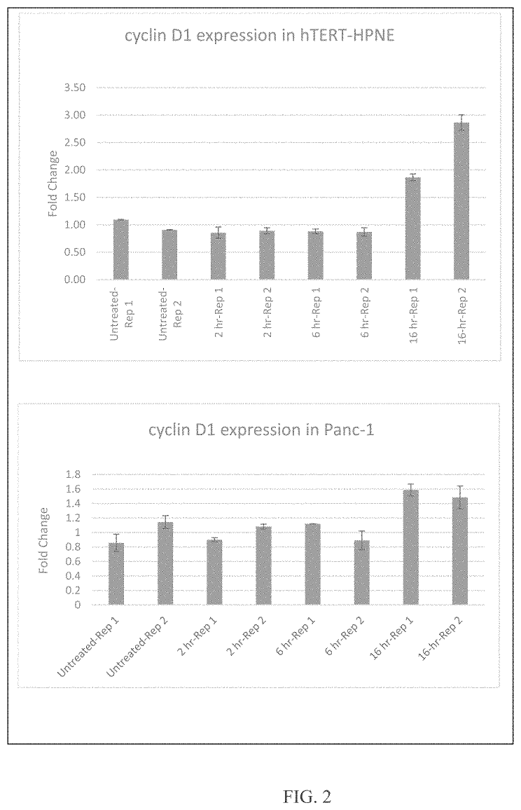

The effects of the composition of EXAMPLE IV on the expression levels of the five genes of interest for the normal (hTERT-HPNE) and cancerous (Panc-1) pancreatic cell lines are shown in FIGS. 2-7. The data are reported as the fold change relative to the untreated control.

The expression of cyclin D1 (FIG. 2) drops slightly from 2-6 hours with treatment of The composition of EXAMPLE IV before rising close to 2 to 3-fold above the untreated level by hour 16 for the normal (hTERT-HPNE) cell line. For the cancer cell line (Panc-1), cyclin D1 largely remains unchanged for the treated cells relative to the untreated controls out to 6 hours before increasing to 140 to 160% of the levels of the untreated groups.

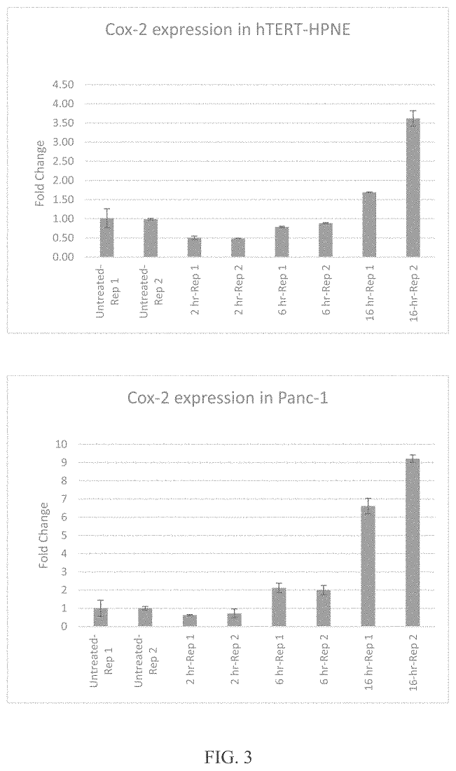

The expression of Cox-2 in response to treatment with the composition of EXAMPLE IV for the normal (hTERT-HPNE) and cancer (Panc-1) cell lines is shown in FIG. 3. For hTERT-HPNE, Cox-2 expression decreases to 50% of the untreated control by two hours. The levels of Cox-2 mRNA climb to 80-90% of the untreated control by six hours and rebound to 1.5 to 3.6 times that of untreated by 16 hours. The trend is similar for Panc-1 with the rebound exceeding the untreated levels by 6 hours and reaching levels 660 to 920% of that of the untreated group.

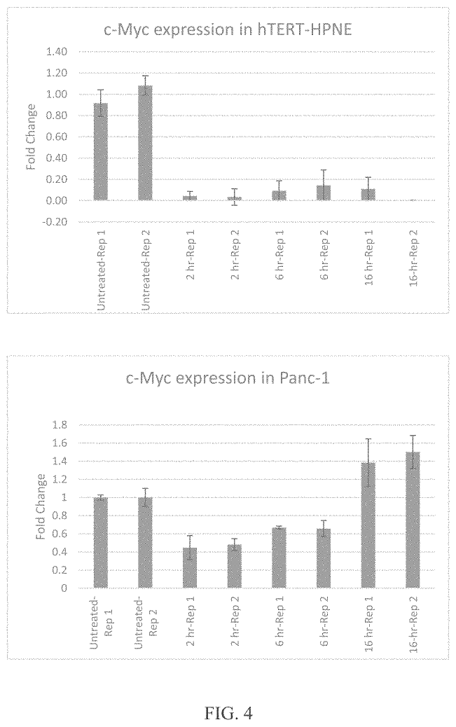

The expression of c-Myc in response to treatment with the composition of EXAMPLE IV for the normal (hTERT-HPNE) and cancer (Panc-1) cell lines is shown in FIG. 4. For hTERT-HPNE, c-Myc levels drop to less than 5% of the untreated levels by 2 hours and remain at less than 15% of untreated levels through hour sixteen. For Panc-1, a less dramatic reduction is observed at the two hour time point where c-Myc mRNA levels are from 44 to 48% of those of the untreated control. The mRNA levels trend upward by hour six and rebound past the untreated levels to 138 to 150% of the untreated controls.

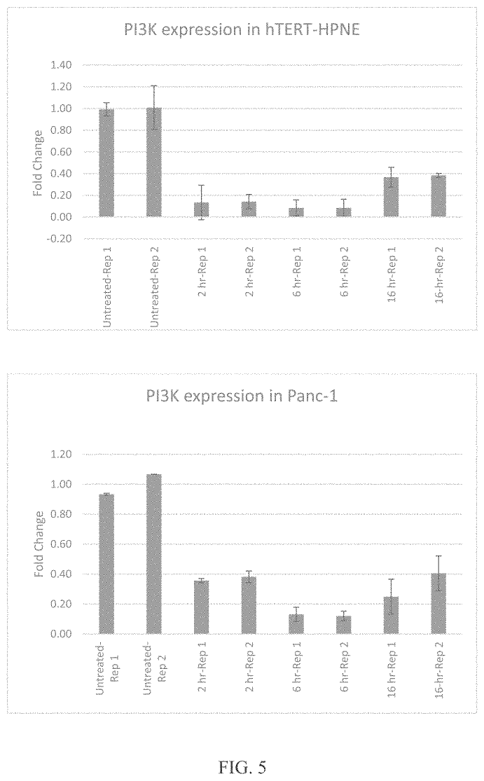

The expression of PI3K in response to treatment with the composition of EXAMPLE IV for the normal (hTERT-HPNE) and cancer (Panc-1) cell lines is shown in FIG. 5. The trends are similar for both cell lines with substantial reductions in mRNA levels by two hours and decreasing further out to six hours before trending upwards, but still significantly reduced relative to the untreated groups by 16 hours.

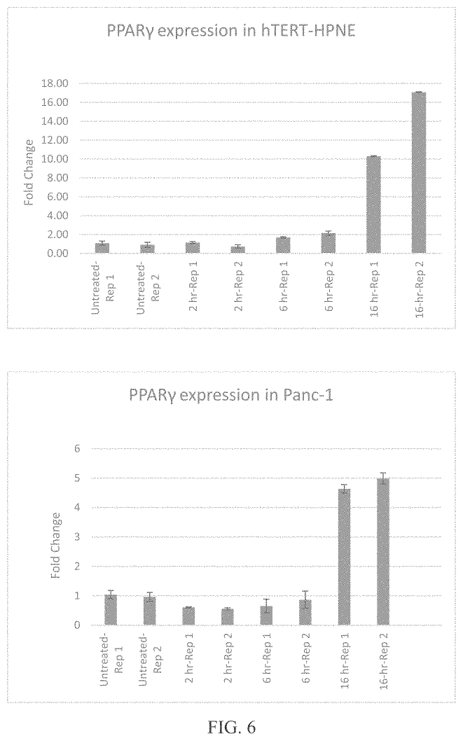

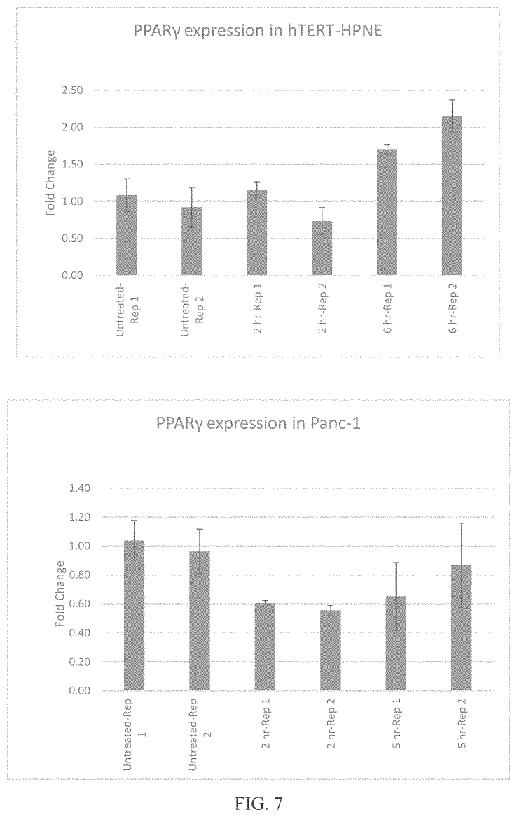

The expression of PPARg in response to treatment with the composition of EXAMPLE IV for the normal (hTERT-HPNE) and cancer (Panc-1) cell lines is shown in FIGS. 6 and 7. FIG. 6 shows the results for all treatments, while the 16 hour time point is left off of FIG. 7 for clarity. For hTERT-HPNE, the data is not clear but possibly there is a small drop in PPARg mRNA levels at two hours FIGS. 6 and 7 top frames. However, at six hours mRNA levels are 1.5 to 2.3-fold above the untreated levels and reach levels exceeding 10 times that of the untreated by 16 hours. For Panc-1, PPARg mRNA levels are clearly reduced to 60% of the untreated levels. By six hours, levels are approaching those of the untreated group and exceeding those by 6-fold by 16 hours.

It should be noted that the Ct values for all the target genes in the QPCR amplified between 20 and 30 cycles, except for Cox-2 and PPARg, which averaged 33-35 cycles. Therefore, all the genes would be considered expressed at moderate levels except for Cox-2 and PPARg, which would be considered low expressed. Low expressed genes are more subject to large fold-change fluctuations between replicates when doing QPCR. This could explain the remarkably high rebound levels observed for Cox-2 and PPARg at 16 hours.

The fold changes are listed numerically in Tables 2 and 3.

TABLE 2
Relative Change of Expression of Genes of Interest for
the composition of EXAMPLE IV Treated hTERT-HPNE.

| | | Fold Change Relative | |
|---|---|---|---|
| Gene Target | Sample | to Untreated | Std. Dev. |
| Cyclin | Untreated-Rep 1 | 1.09 | 0.004 |
| | Untreated-Rep 2 | 0.91 | 0.004 |
| | 2 hr-Rep 1 | 0.85 | 0.103 |
| | 2 hr-Rep 2 | 0.89 | 0.055 |
| | 6 hr-Rep 1 | 0.88 | 0.042 |
| | 6 hr-Rep 2 | 0.87 | 0.076 |
| | 16 hr-Rep 1 | 1.87 | 0.061 |
| | 16-hr-Rep 2 | 2.87 | 0.139 |
| Cox-2 | Untreated-Rep 1 | 1.01 | 0.247 |
| | Untreated-Rep 2 | 0.99 | 0.020 |
| | 2 hr-Rep 1 | 0.50 | 0.043 |
| | 2 hr-Rep 2 | 0.49 | 0.003 |
| | 6 hr-Rep 1 | 0.79 | 0.015 |
| | 6 hr-Rep 2 | 0.88 | 0.016 |
| | 16 hr-Rep 1 | 1.69 | 0.007 |
| | 16-hr-Rep 2 | 3.62 | 0.202 |
| c-Myc | Untreated-Rep 1 | 0.92 | 0.125 |
| | Untreated-Rep 2 | 1.08 | 0.091 |
| | 2 hr-Rep 1 | 0.04 | 0.043 |
| | 2 hr-Rep 2 | 0.03 | 0.078 |
| | 6 hr-Rep 1 | 0.09 | 0.093 |
| | 6 hr-Rep 2 | 0.14 | 0.144 |
| | 16 hr-Rep 1 | 0.11 | 0.110 |
| | 16-hr-Rep 2 | 0.00 | 0.003 |
| PI3K | Untreated-Rep 1 | 0.99 | 0.061 |
| | Untreated-Rep 2 | 1.01 | 0.201 |
| | 2 hr-Rep 1 | 0.13 | 0.160 |
| | 2 hr-Rep 2 | 0.14 | 0.067 |
| | 6 hr-Rep 1 | 0.08 | 0.074 |
| | 6 hr-Rep 2 | 0.08 | 0.080 |
| | 16 hr-Rep 1 | 0.37 | 0.091 |
| | 16-hr-Rep 2 | 0.38 | 0.019 |
| PPAR g | Untreated-Rep 1 | 1.08 | 0.219 |
| | Untreated-Rep 2 | 0.92 | 0.267 |
| | 2 hr-Rep 1 | 1.15 | 0.104 |
| | 2 hr-Rep 2 | 0.73 | 0.182 |
| | 6 hr-Rep 1 | 1.70 | 0.064 |
| | 6 hr-Rep 2 | 2.15 | 0.215 |
| | 16 hr-Rep 1 | 10.3 | 0.048 |
| | 16-hr-Rep 2 | 17.1 | 0.046 |

TABLE 3
Relative Change of Expression of Genes of Interest
for the composition of EXAMPLE IV Treated Panc-1.

| | | Fold Change Relative | |
|---|---|---|---|
| Gene Target | Sample | to Untreated | Std. Dev. |
| Cyclin | Untreated-Rep 1 | 0.86 | 0.119 |
| | Untreated-Rep 2 | 1.14 | 0.089 |
| | 2 hr-Rep 1 | 0.90 | 0.028 |
| | 2 hr-Rep 2 | 1.08 | 0.034 |
| | 6 hr-Rep 1 | 1.12 | 0.001 |
| | 6 hr-Rep 2 | 0.89 | 0.129 |
| | 16 hr-Rep 1 | 1.59 | 0.082 |
| | 16-hr-Rep 2 | 1.48 | 0.158 |
| Cox-2 | Untreated-Rep 1 | 1.00 | 0.443 |
| | Untreated-Rep 2 | 1.00 | 0.096 |
| | 2 hr-Rep 1 | 0.62 | 0.034 |
| | 2 hr-Rep 2 | 0.72 | 0.239 |
| | 6 hr-Rep 1 | 2.11 | 0.260 |
| | 6 hr-Rep 2 | 2.00 | 0.252 |
| | 16 hr-Rep 1 | 6.61 | 0.424 |
| | 16-hr-Rep 2 | 9.21 | 0.202 |
| c-Myc | Untreated-Rep 1 | 1.00 | 0.029 |
| | Untreated-Rep 2 | 1.00 | 0.101 |
| | 2 hr-Rep 1 | 0.45 | 0.133 |
| | 2 hr-Rep 2 | 0.48 | 0.066 |
| | 6 hr-Rep 1 | 0.67 | 0.016 |
| | 6 hr-Rep 2 | 0.66 | 0.089 |
| | 16 hr-Rep 1 | 1.38 | 0.262 |
| | 16-hr-Rep 2 | 1.50 | 0.182 |
| PI3K | Untreated-Rep 1 | 0.93 | 0.006 |
| | Untreated-Rep 2 | 1.07 | 0.001 |
| | 2 hr-Rep 1 | 0.36 | 0.014 |
| | 2 hr-Rep 2 | 0.38 | 0.038 |
| | 6 hr-Rep 1 | 0.13 | 0.048 |
| | 6 hr-Rep 2 | 0.12 | 0.032 |
| | 16 hr-Rep 1 | 0.25 | 0.117 |
| | 16-hr-Rep 2 | 0.41 | 0.116 |
| PPAR g | Untreated-Rep 1 | 1.04 | 0.140 |
| | Untreated-Rep 2 | 0.96 | 0.154 |
| | 2 hr-Rep 1 | 0.61 | 0.015 |
| | 2 hr-Rep 2 | 0.56 | 0.034 |
| | 6 hr-Rep 1 | 0.65 | 0.234 |
| | 6 hr-Rep 2 | 0.87 | 0.292 |
| | 16 hr-Rep 1 | 4.63 | 0.144 |
| | 16-hr-Rep 2 | 4.98 | 0.192 |

Human Phosphorylation Pathway Profiling. Human phosphorylation profiling was carried out to determine the relative levels of phosphorylated proteins in five important cellular signaling pathways including the MAPK, AKT, JAK/STAT, NFkb and TGFb pathways. The assay was performed using Ray Biotech's Human Phosphorylation Pathway Profiling Array C55 kit which is an immuno-sandwich style array. Capture antibodies specific to each phosphoprotein of interest are coupled to a membrane. The capture antibodies are coupled to duplicate spots on each array for every protein of interest, and in quadruplicate for positive controls on some of the arrays. The proteins of interest from a cell lysate are subsequently captured by the membrane-bound antibody. A cocktail of detection antibodies specific to each of the proteins of interest is reacted with the membrane. Finally, an HRP-conjugated antibody specific for the detection antibodies is reacted with the membrane to allow chemiluminescent detection of each of the proteins of interest. The membrane arrays have various control spots that allow the signals from the individual spots to be normalized, and to allow normalization between arrays that received lysates from different treatments.

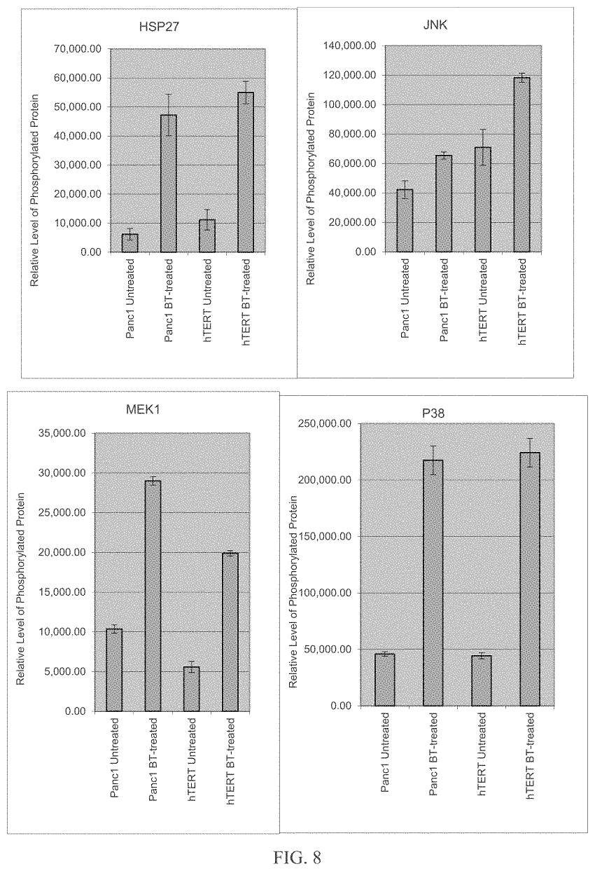

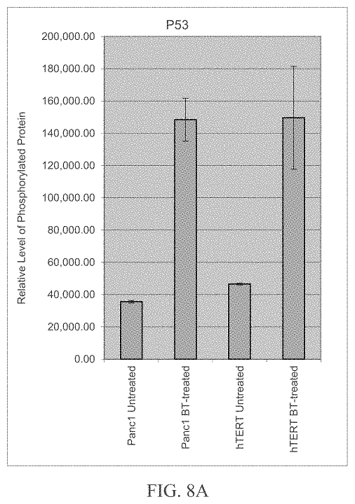

hTERT-HPNE and Panc-1 cells, untreated and the composition of EXAMPLE IV-treated (100 ug/mL, 2 hours) were harvested, cell lysates were prepared, and the cell lysates were incubated with the profiling arrays. The arrays were worked up as described in the Materials and Methods section. The results are shown in FIGS. 8-20. The protein symbols for the proteins examined in the five different cell signaling pathways are listed in Table 4. Links to the full description of the protein at the Universal Protein Resource (UniProt) database can be found at https://www.raybiotech.com/human-phosphorylation-pathway-profiling-array-c55/.

TABLE 4
Pathways and phosphoprotein targets examined
by phosphoprotein array assay.

| MAPK | AKT | JAK/STAT | NFkb | TGFb |
|---|---|---|---|---|
| CREB | AKT | EGFR | ATM | ATF2 |
| Erk1/2 | AMPKa | JAK1 | eIF2a | c-Fos |
| HSP27 | BAD | JAK2 | HDAC2 | c-Jun |
| JNK | 4E-BP1 | SHP1 | HDAC4 | Smad1 |
| Mek1 | GSk3a | SHP2 | IkBa | Smad2 |
| MKK3 | GSK3b | Src | MSK1 | Smad4 |
| MKK6 | mTor | Stat1 | NFkb | Smad5 |
| MSK2 | P27 | Stat2 | TAK1 | |
| P38 | P70S6k | Stat3 | TBK1 | |
| P53 | PDK1 | Stat5 | ZAP70 | |
| RSK1 | PRAS40 | Stat6 | | |
| RSK2 | PTEN | TYK2 | | |
| | Raf-1 | | | |
| | RPS6 | | | |

MAPK cell signaling pathway results. For the MAPK cell signaling pathway, the levels of phosphoproteins HSP27, JNK, MEK1, P38 and P58 were all found to increase in response to treatment with the composition of EXAMPLE IV for both the normal and cancer cell lines (FIG. 8).

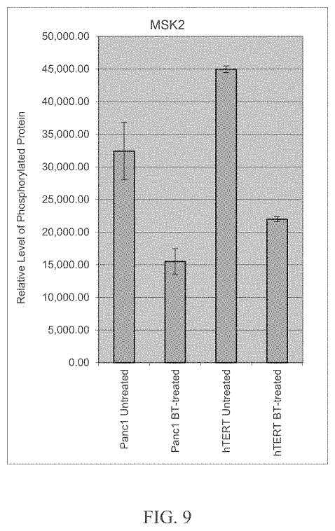

In contrast, treatment of both cell lines with the composition of EXAMPLE IV resulted in comparable decreases in phosphorylated MSK2 for both cell lines (FIG. 9).

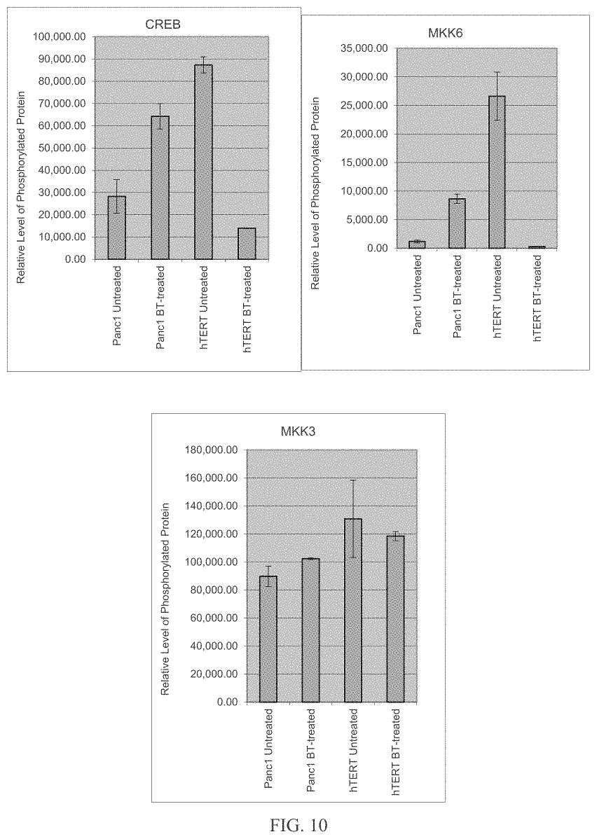

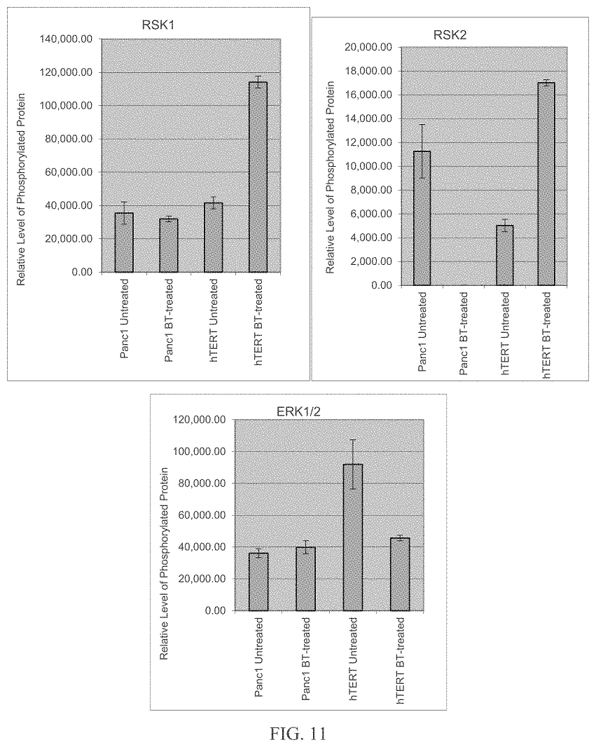

The responses for the remaining MAPK targets are mixed. Levels of phosphorylated CREB and MKK6, and possibly MKK3, increased in Panc-1 cells, while it decreased for hTERT-HPNE cells treated with the composition of EXAMPLE IV (FIG. 10).

The levels of phosphorylated RSK 1 and RSK2 increased relative to untreated cells for hTERT-HPNE, while the levels were unaffected in Panc-1 cells or dropped below the level of detection for RSK2 (FIG. 11). Finally, phosphorylated ERK½ decreased in response to the composition of EXAMPLE IV treatment for hTERT-HPNE cells while remaining unchanged in Panc-1 cells (FIG. 11).

The results for the MAPK pathway are summarized in Table 5 in which the fold change in phosphorylated proteins, relative to untreated, is shown for both Panc-1 and hTERT-HPNE cells.

TABLE 5
Change in level of MAPK pathway phosphoproteins
relative to the untreated cells.

| | |
|---|---|
| Panc-1 | hTERT-HPNE |

| Protein | Fold Change | Protein | Fold Change |
|---|---|---|---|
| HSP27 | 7.7 | P38 | 5.06 |
| MKK6 | 7.3 | HSP27 | 4.93 |
| P38 | 4.7 | IC | 4.27 |
| P53 | 4.2 | Mek1 | 3.56 |
| IC | 4.1 | RSK2 | 3.39 |
| Mek1 | 2.8 | P53 | 3.21 |
| IC | 2.7 | RSK1 | 2.75 |
| CREB | 2.3 | IC | 2.29 |
| IC | 2.2 | JNK | 1.67 |
| JNK | 1.5 | IC | 1.60 |
| MKK3 | 1.1 | MKK3 | 0.91 |
| Erk1/2 | 1.1 | IC | 0.73 |
| IC | 1.0 | Erk1/2 | 0.50 |
| RSK1 | 0.9 | MSK2 | 0.49 |
| MSK2 | 0.5 | CREB | 0.16 |
| RSK2 | 0.0 | MKK6 | 0.01 |

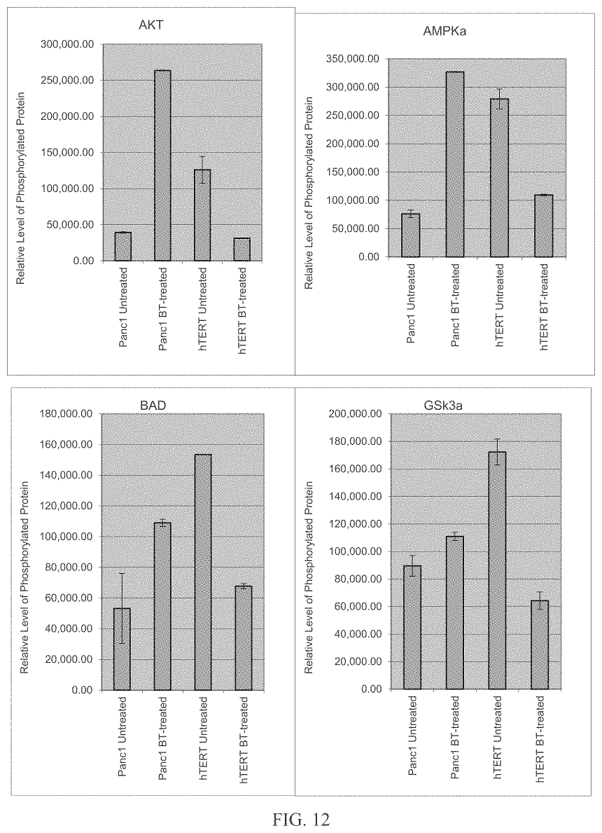

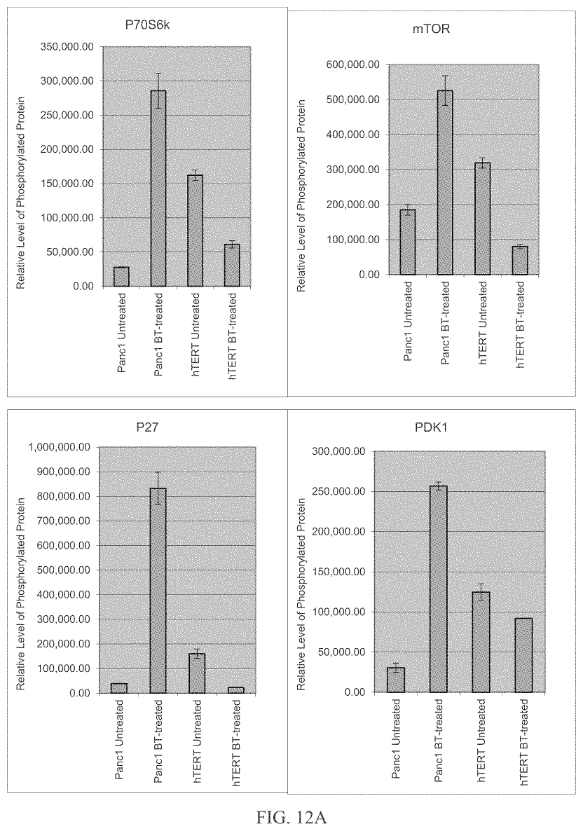

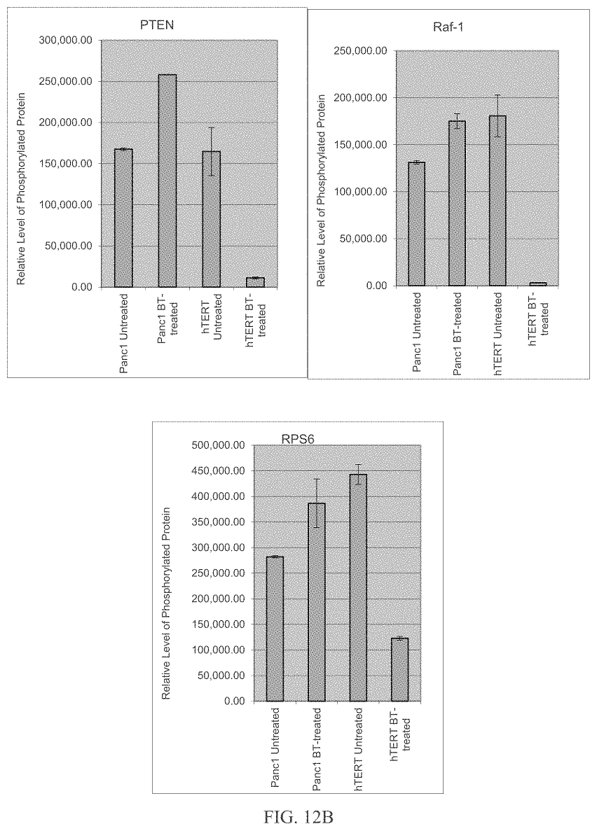

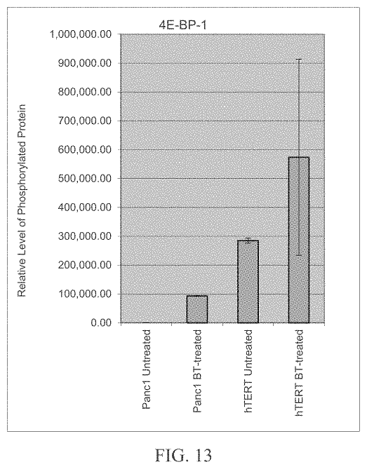

AKT cell signaling pathway results. For the AKT cell signaling pathway, the effect of the composition of EXAMPLE IV on the levels of phosphoproteins was quite different. The trend that was observed was that in response to The composition of EXAMPLE IV treatment, the levels of AKT pathway phosphoproteins increased for the Panc-1 cells while they decreased for the hTERT-HPNE cells for all but one of the targets including: AKT, AMPKa, BAD, GSk3a, GSk3b, mTOR, P27, P70S6k, PDK1, PRAS40, PTEN, Raf-1, and RPS6 (FIG. 12). The only exception was for 4E-BP1, in which phosphor-4E-BP-1 increased for both cell lines in response to the composition of EXAMPLE IV treatment (FIG. 13).

The results for the AKT pathway are summarized in Table 6 in which the fold change in phosphorylated proteins, relative to untreated, is shown for both Panc-1 and hTERT-HPNE cells.

TABLE 6
Change in level of AKT pathway phosphoproteins
relative to the untreated cells.

| | |
|---|---|
| Panc-1 | hTERT-HPNE |

| Protein | Fold Change | Protein | Fold Change |
|---|---|---|---|
| P27 | 21.7 | IC | 12.15 |
| IC | 14.9 | 4E-BP1 | 2.01 |
| P70S6k | 10.3 | PDK1 | 0.74 |
| PDK1 | 8.4 | BAD | 0.44 |
| AKT | 6.7 | AMPKa | 0.39 |
| AMPKa | 4.3 | P70S6k | 0.38 |
| mTor | 2.8 | GSk3a | 0.37 |
| IC | 2.3 | IC | 0.34 |
| PRAS40 | 2.1 | GSK3b | 0.28 |
| BAD | 2.0 | RPS6 | 0.28 |
| PTEN | 1.5 | mTor | 0.25 |
| GSK3b | 1.4 | AKT | 0.25 |
| RPS6 | 1.4 | PRAS40 | 0.23 |
| Raf-1 | 1.3 | P27 | 0.14 |
| GSk3a | 1.2 | PTEN | 0.07 |
| 4E-BP1 | | Raf-1 | 0.02 |

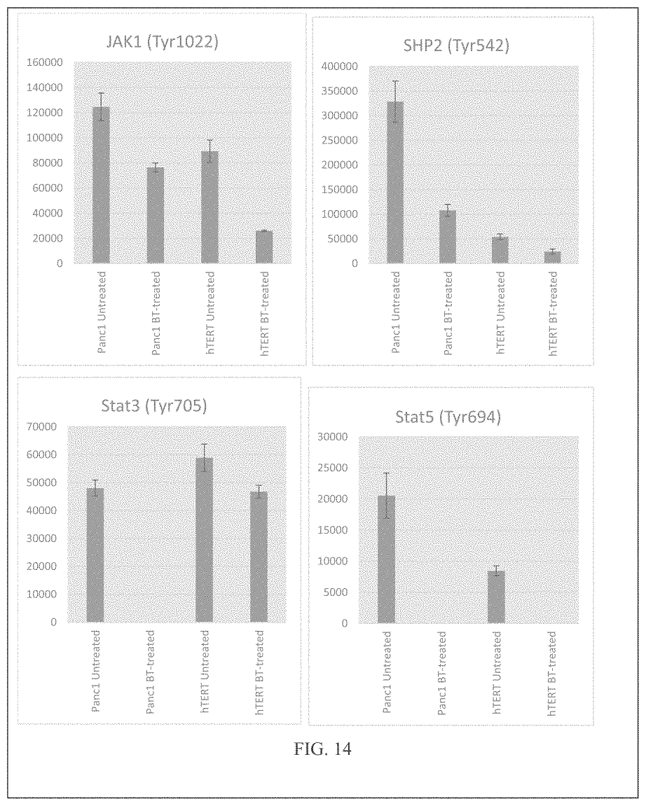

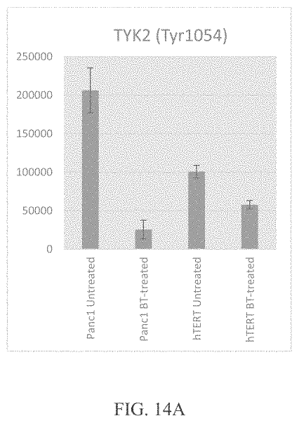

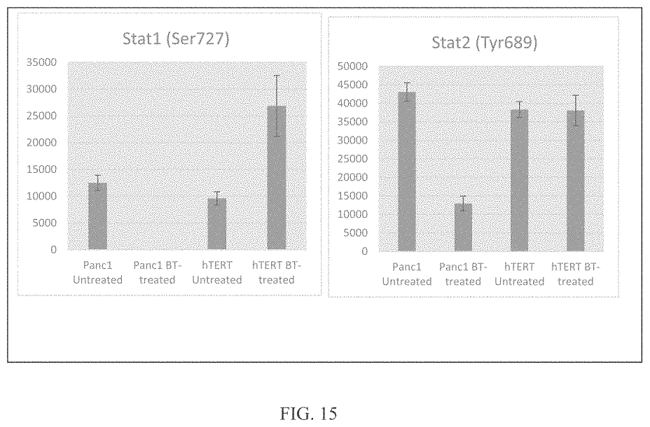

JAK/STAT cell signaling pathway results. For the JAK/STAT cell signaling pathway, the pattern of response for the change in phosphoproteins with the composition of EXAMPLE IV treatment was the same for both cell lines. For JAK1, SHP2, STAT3, STAT5 and TYK2, the levels of the phosphorylated protein decreased in response to the composition of EXAMPLE IV for both cell lines (FIG. 14 and FIG. 14A). The exceptions were Stat1 and Stat2 in which the phosphoprotein levels dropped for Panc-1 while they increased or were unchanged for hTERT-HPNE for Stat1 or Stat2, for the treated relative to untreated cells (FIG. 15).

For EGFR, JAK2, SHP1, Src and STAT6, the data was uninformative as the levels of detection were below background for these phosphoproteins.

The results for the JAK/STAT pathway are summarized in Table 7 in which the fold change in phosphorylated proteins, relative to untreated, is shown for both Panc-1 and hTERT-HPNE cells.

TABLE 7
Change in level of JAK/STAT pathway phosphoproteins
relative to the untreated cells.

| | |
|---|---|
| Panc-1 | hTERT-HPNE |

| Protein | Fold Change | Protein | Fold Change |
|---|---|---|---|
| JAK1 | 0.61 | Stat1 | 2.80 |
| SHP2 | 0.33 | Stat2 | 0.99 |
| Stat2 | 0.30 | Stat3 | 0.79 |
| TYK2 | 0.12 | TYK2 | 0.57 |
| JAK2 | — | SHP2 | 0.45 |
| SHP1 | — | JAK1 | 0.29 |
| Stat1 | — | Stat5 | — |
| Stat3 | — | EGFR | — |
| Stat5 | — | Src | — |
| Stat6 | — | Stat6 | — |
| EGFR | — | JAK2 | — |
| Src | — | SHP1 | — |

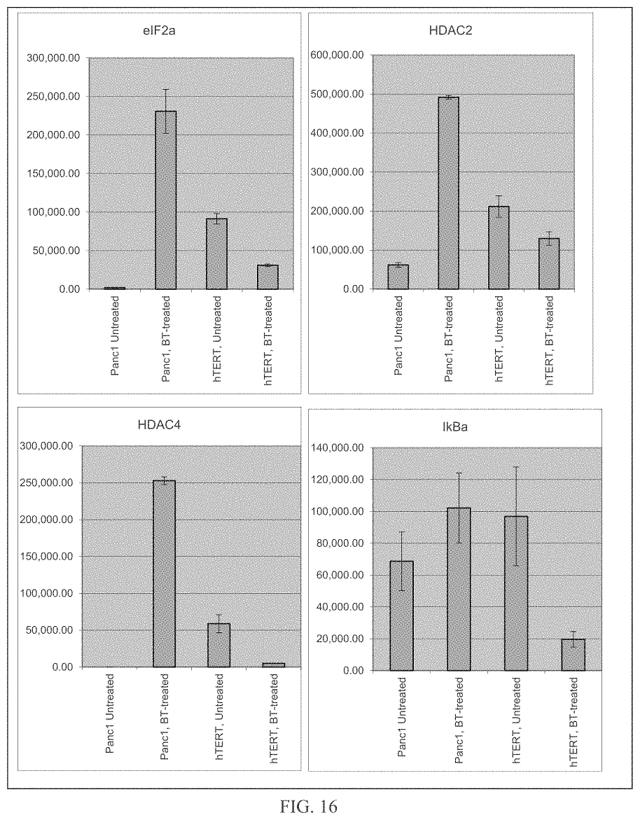

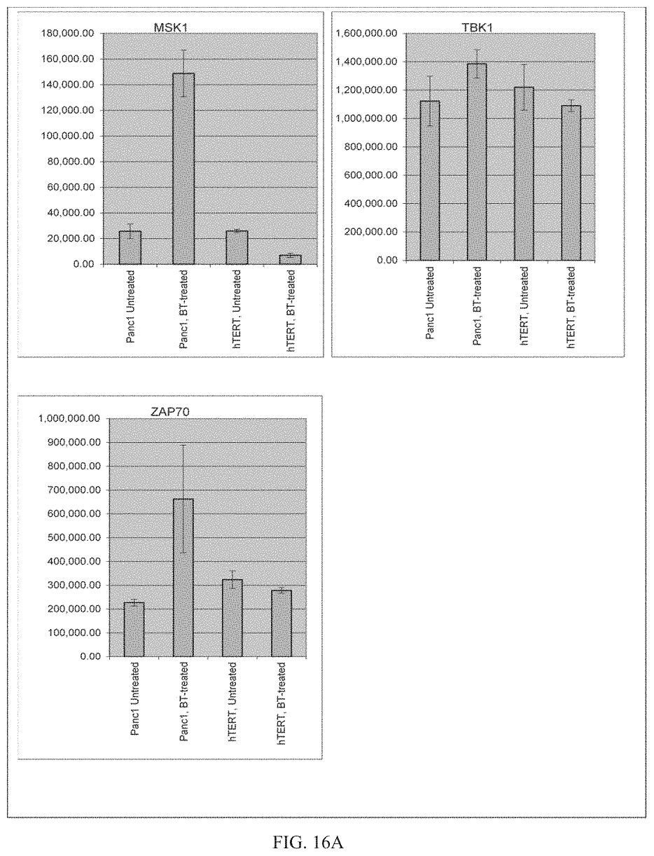

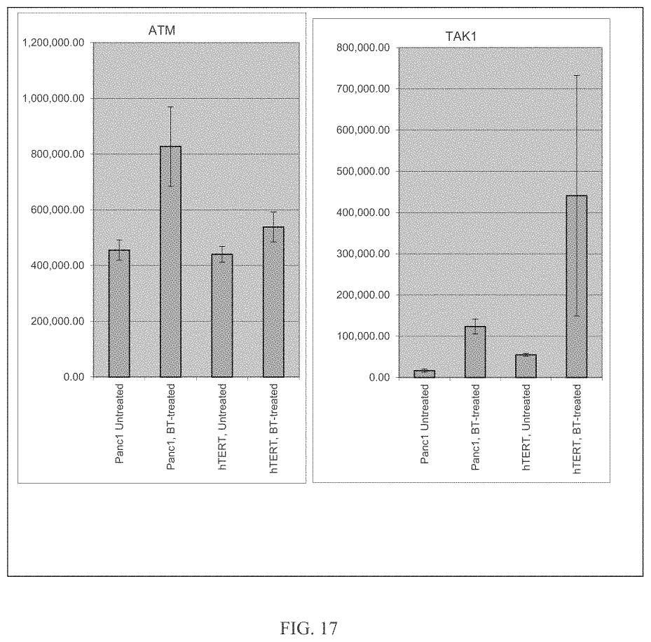

NFkb cell signaling pathway results. For the NFkb cell signaling pathway, the trend for the effect of the composition of EXAMPLE IV on the levels of phosphoproteins was largely that they increased for Panc-1 cells and decreased for hTERT-HPNE in response to the composition of EXAMPLE IV treatment. This was true for eIF2a, HDAC2, HDAC4, IkBa, MSK1, TBK1 and ZAP70 (FIG. 16). For ATM and TAK1, the composition of EXAMPLE IV treatment produced an increase in the phosphoprotein for both cell lines (FIG. 17). Only NFkb was uninformative, as the levels of phosphorylated NFkb were below background levels for all conditions.

The results for the NFkb pathway are summarized in Table 8 in which the fold change in phosphorylated proteins, relative to untreated, is shown for both Panc-1 and hTERT-HPNE cells.

TABLE 8
Change in level of NFkb pathway phosphoproteins
relative to the untreated cells.

| | |
|---|---|
| Panc-1 | hTERT-HPNE |

| Protein | Fold Change | Protein | Fold Change |
|---|---|---|---|
| ATM | 1.8 | ATM | 1.22 |
| eIF2a | 117.9 | TAK1 | 8.04 |
| HDAC2 | 8.0 | TBK1 | 0.89 |
| TAK1 | 7.4 | ZAP70 | 0.86 |
| MSK1 | 5.8 | HDAC2 | 0.61 |
| ZAP70 | 2.9 | eIF2a | 0.34 |
| IkBa | 1.5 | MSK1 | 0.27 |
| TBK1 | 1.2 | IkBa | 0.20 |
| HDAC4 | — | HDAC4 | 0.08 |
| NFkB | — | NFkB | — |

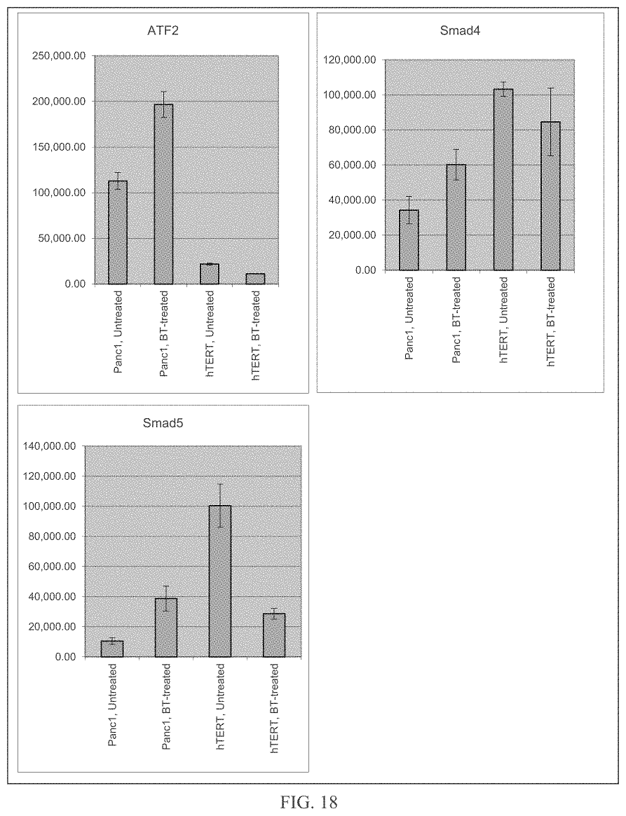

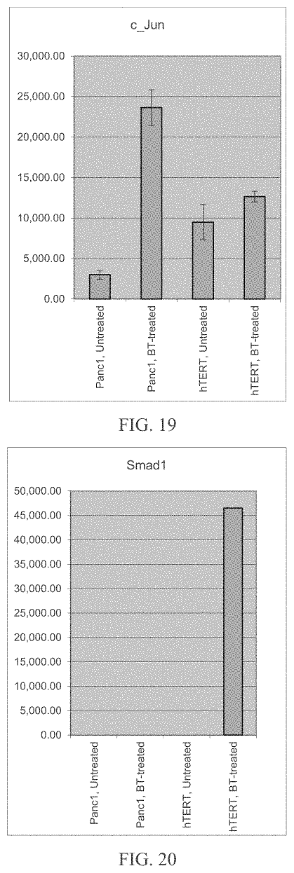

TGFb cell signaling pathway results. For the TGFb cell signaling pathway, the pattern of response for the change in phosphoproteins was largely that the phosphoproteins increased and decreased for Panc-1 and hTERT-HPNE, respectively, in response to the composition of EXAMPLE IV treatment for ATF2, Smad4 and Smad5 (FIG. 18). For c-Jun, the levels of the phosphorylated protein increased in response to the composition of EXAMPLE IV treatment for both cell lines (FIG. 19). Phosphorylated Smad-1 levels were increased for hTERT-HPNE in response to the composition of EXAMPLE IV, but since the untreated levels were below detection, the amount of stimulation is not evident (FIG. 20). Phosphorylated Smad-1 levels for Panc-1 were undetectable for both treated and untreated cells, while the levels of phosphorylated c-Fos were below detectable levels for both cell lines regardless of treatment.

The results for the TGFb pathway are summarized in Table 9, in which the fold change in phosphorylated proteins, relative to untreated, is shown for both Panc-1 and hTERT-HPNE cells.

TABLE 9
Change in level of TGFb pathway phosphoproteins
relative to the untreated cells.

| | |
|---|---|
| Panc-1 | hTERT-HPNE |

| Protein | Fold Change | Protein | Fold Change |
|---|---|---|---|
| c-Jun | 7.9 | c-Jun | 1.33 |
| Smad5 | 3.7 | smad4 | 0.82 |
| smad4 | 1.8 | ATF2 | 0.51 |
| ATF2 | 1.7 | Smad5 | 0.29 |
| c-Fos | — | c-Fos | — |
| Smad1 | — | Smad1 | — |
| Smad2 | — | Smad2 | — |

Conclusions: The effect of the composition of EXAMPLE IV on the transcriptional levels of cyclin-D1, Cox-2, c-Myc, PI3K and PPARg by qRT-PCR for a normal (hTERT-HPNE) and a cancer (Panc-1) pancreatic cell line was evaluated. Additionally, the effect of The composition of EXAMPLE IV on the level of phosphorylated proteins in five human cell signaling pathways including the MAPK, AKT, JAK/STAT, NFkb and TGFb pathways was evaluated using a Human Phosphorylation Pathway Profiling Array.

Some notable outcomes from this study follow:

The composition of EXAMPLE IV is non-toxic and was well tolerated by both cell lines even at the highest concentrations examined.

The composition of EXAMPLE IV largely elicited reductions in mRNA levels for cyclin-D1, Cox-2, c-Myc, PIK3 and PPARg for both cell lines.

The effect of the composition of EXAMPLE IV on the levels of key cell signaling phosphoproteins in five important signaling pathways varied by pathway. For the MAPK pathway, the normal and cancer cell lines largely responded in the same direction to the composition of EXAMPLE IV. For the AKT pathway, key phosphoproteins largely increased in level for the cancer cell line (Panc-1), while for the normal cell line (hTERT-HPNE) they decreased in response to the composition of EXAMPLE IV. For the JAK/STAT pathway, both cell lines largely responded to the composition of EXAMPLE IV with decreases in the levels of phosphorylation for key phosphoproteins. The exception being for Stat1 and Stat2. For NFkb cell signaling, the cell lines responded reciprocally to the composition of EXAMPLE IV with increases in levels of phosphorylation for Panc-1 and decreases for hTERT-HPNE. Finally, for TGFb cell signaling, the inverse was largely true with increased and decreased phosphorylation for Panc-1 and hTERT-HPNE, respectively.

### EXAMPLE VII

Ability of the described compositions to regulate biosignaling cascades in human subjects

Introduction: This study was conducted to determine if a composition as described herein (in this case, the composition of Example IV) could restore homeostasis by re-equilibrating the signaling mechanisms of the body. The purpose of this study was to investigate the ability of the described compositions to regulate biosignaling cascade reactions in human subjects.

Methods: To test the ability of the compositions in regulating various signaling mechanisms, 15 individuals were administered daily dosages of the composition of Example IV for three weeks. During the test period, the participants' saliva samples were collected at days 0, 7, and 21 to measure change of various salivary biomarkers (e.g., cortisol, interleukins, and TNF-α). Outliers were calculated and removed by using an outlier calculator available at miniwebtool.com/outlier-calculator/.

Results:

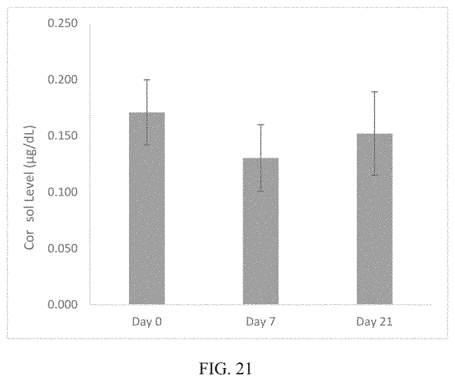

The composition reduced salivary cortisol levels: To evaluate how the composition of Example IV affects stress levels, salivary cortisol levels were monitored in this study. Salivary cortisol concentrations and stress levels are positively correlated. People who experience higher stress also have increased cortisol levels in saliva¹. Ishisaka et al. infra. Additionally, cortisol is a master regulator in many biosignaling pathways in the body; higher cortisol levels in the body indicates that several biosignaling cascades are out of balance. In this study, it was observed that the mean cortisol level of the 15 participants was decreased on days 7 and 21 after taking the composition of Example IV (FIG. 21). This decreasing trend indicates that the composition may reduce daily stress levels and improve biosignaling by regulating cortisol levels in the body.

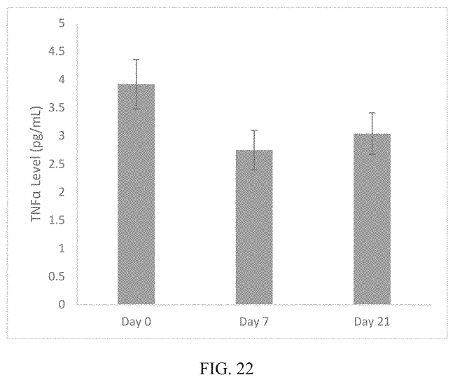

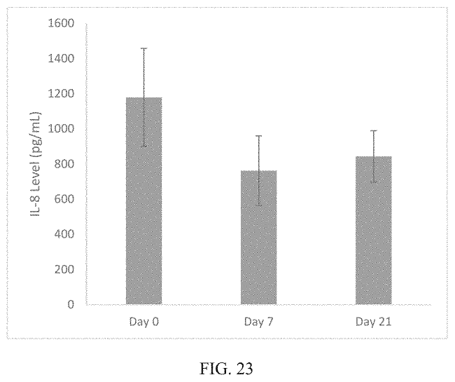

The composition reduced inflammation by mediating TNF-α and IL-8 levels: In addition to stress, chronic inflammation is another common cause of poor quality of life. Therefore, various inflammation markers and cytokine levels secreted in saliva were also monitored. Of particular interest was the cytokine Tumor Necrosis Factor alpha (“TNF-α”), which is a well-known inflammatory marker and another indicator of the health of different biosignaling reactions. Interestingly, mean TNF-α level was significantly reduced, 30% on days 7 and 23% on day 21, when compared to day 0 (FIG. 22). Similarly, another pro-inflammatory cytokine called interleukin 8 (IL-8) also showed a 35% and 28% reduction on days 7 and 21, respectively, when compared to day 0 (FIG. 23). The combined data presented herein suggests that the composition has a potent anti-inflammatory affect and should improve daily quality of life by reducing cellular inflammation. Reduction of inflammation in the body can improve many facets of human health including mood, cognitive function, and natural energy.

Conclusion: The described compositions have the ability to regulate biosignaling cascades in human subjects related to stress, inflammation, and other biosignaling pathways that pool into cortisol and TNF-α levels. These results validate and strengthen other data demonstrating the ability of the compositions to regulate biosignaling mechanisms in human cell cultures. Both in vitro and in vivo clinical data support the biosignaling mode of action and efficacy of the compositions.

### REFERENCES

(The contents of each of which are incorporated herein by this reference.)

Diwaker et al., “Rhodiola inhibits dengue virus multiplication by inducing innate immune response genes RIG-I, MDA5 and ISG in human monocytes,” Archives of Virology vol. 159, pp. 1975-1986 (2014).

Feng et al., “Ginkgolide B ameliorates oxidized low-density lipoprotein-induced endothelial dysfunction via modulating Lectin-like ox-LDL-receptor-1 and NADPH oxidase 4 expression and inflammatory cascades,” Phytotherapy Research—Wiley Online Library 2018; https://onlinelibrary.wiley.com/doi/abs/10.1002/ptr.6177.

Gerbino et al., “Dandelion Root Extract Induces Intracellular Ca2+ Increases in HEK293 Cells,” Int. J. Mol. Sci. 2018, 19, 1112; doi:10.3390/ijms19041112.

Huang et al., “Berberine inhibits 3T3-L1 adipocyte differentiation through the PPARy pathway,” Biochemical and Biophysical Research Communications 348 (2006) 571-578.

Ishisaka, A. et al. Association of Salivary Levels of Cortisol and Dehydroepiandrosterone With Periodontitis in Older Japanese Adults. J Periodontol 78, 1767-1773 (2007).

Lee et al., “Berberine, a Natural Plant Product, Activates AMP-Activated Protein Kinase With Beneficial Metabolic Effects in Diabetic and Insulin-Resistant States,” Diabetes 55:2256-2264, (2006).

Lee et al., “Arctiin is a pharmacological inhibitor of STAT3 phosphorylation at tyrosine 705 residue and potentiates bortezomib-induced apoptotic and anti-angiogenic effects in human multiple myeloma cells,” Phytomedicine Volume 55,1 March 2019, Pages 282-292.

Neergheen et al., “Targeting specific cell signaling transduction pathways by dietary and medicinal phytochemicals in cancer chemoprevention,” Toxicology 278:229-241 (2010).

Singh et al., “Misconstrued versatility of Ganoderma lucidum: a key player in multitargeted cellular signaling,” Tumor Biology volume 37, pages 2789-2804 (2016).

Vendrame et al., “Anti-inflammatory effect of anthocyanins via modulation of nuclear factor-jB and mitogen-activated protein kinase signaling cascades,” Nutrition Reviews VR Vol. 73(6):348-358 (2015).

Yang et al., “Curcumin induces the apoptosis of human monocytic leukemia THP-1 cells via the activation of JNK/ERK Pathways,” BMC Complementary and Alternative Medicine 2012, 12:22.

Yang et al., “A novel PTP1B inhibitor extracted from Ganoderma lucidum ameliorates insulin resistance by regulating IRS1-GLUT4 cascades in the insulin signaling pathway,” Food Funct., 2018, 9, 397-406; DOI: 10.1039/C7FO01489A.

What is claimed is:

1. A dosage form for administration to a subject, the dosage form comprising at least eight phytonutrients selected from the group consisting of resveratrol, berberine, epigallocatechin gallate, quercetin, dandelion root extract powder, curcuminoids, burdock root powder, Reishi mushroom extract powder, African mango seed extract, rosavins, ginkgolides, ellagic acid, Ginsengosides, and gingerols.

2. The dosage form of claim 1, wherein the dosage form contains amounts of the selected at least eight phytonutrients to maintain health of at least one generally conserved signal transduction cascade (“STC”) in the subject when the subject ingests at least one of the dosage forms on a daily basis.

3. The dosage form of claim 1, wherein the selected at least eight phytonutrients, when present, are present in the following amounts:
from about 50 to about 250 milligrams of resveratrol,
from about 100 to about 500 milligrams of berberine,
from about 100 to about 400 milligrams of epigallocatechin gallate,
from about 20 to about 100 milligrams of quercetin,
from about 100 to about 300 milligrams of dandelion root extract powder,
from about 20 to about 80 milligrams of curcuminoids,
from about 75 to about 300 milligrams of burdock root powder,
from about 50 to about 200 milligrams of Reishi mushroom extract powder,
from about 75 to about 450 milligrams of African mango seed extract,
from about 2 to about 10 milligrams of Rosavins,
from about 20 to about 60 milligrams of Ginkgolides,
from about 50 to about 110 milligrams ellagic acid,
from about 5 to about 25 milligrams of Ginsengosides, and
from about 2 to about 20 milligrams of Gingerols.

4. The dosage form of claim 1, wherein the at least eight phytonutrients are selected from the group consisting of resveratrol, berberine, epigallocatechin gallate, quercetin, curcuminoids, burdock root powder, rosavins, ginkgolides, and ellagic acid.

5. The dosage form of any one of claims 1, wherein the dosage form is selected from the group consisting of a softgel, capsule, tablet, gel, powder, gummy, liquid, effervescent, bar, topical patch, serum, lotion, and cream.

6. A method of treating a subject to maintain at least one generally conserved signal transduction cascade (“STC”), the method comprising:
administering the dosage form of claim 2 to the subject on a regular basis.

7. A method of treating a subject to maintain at least one generally conserved signal transduction cascade (“STC”), the method comprising:
administering a combination of at least eight selected phytonutrients to the subject, wherein the at least eight phytonutrients are selected are from the group consisting of resveratrol, berberine, epigallocatechin gallate, quercetin, dandelion root extract powder, curcuminoids, burdock root powder, Reishi mushroom extract powder, African mango seed extract, rosavins, ginkgolides, ellagic acid, Ginsengosides, and gingerols.

8. The method according to claim 7, wherein the subject ingests the selected at least eight phytonutrients, when selected, in the following amounts on a daily basis:
from about 50 to about 250 milligrams of resveratrol,
from about 100 to about 500 milligrams of berberine,
from about 100 to about 400 milligrams of epigallocatechin gallate,
from about 20 to about 100 milligrams of quercetin,
from about 100 to about 300 milligrams of dandelion root extract powder,
from about 20 to about 80 milligrams of curcuminoids,
from about 75 to about 300 milligrams of burdock root powder,
from about 50 to about 200 milligrams of Reishi mushroom extract powder,
from about 75 to about 450 milligrams of African mango seed extract,
from about 2 to about 10 milligrams of Rosavins,
from about 20 to about 60 milligrams of Ginkgolides,
from about 50 to about 110 milligrams ellagic acid,
from about 5 to about 25 milligrams of Ginsengosides, and
from about 2 to about 20 milligrams of Gingerols.

9. The method according to claim 6, for use in reducing interference with an STC caused by at least one environmental factor to which the subject is subjected.

10. The method according to any claim 9, wherein the STC is selected from the group consisting of TGF-Beta/Smad2, Bcl2/BAX/P53, TGF-Beta/JNK, iNOS, NADPH Oxidase 4, WNT/Beta-Catenin, STAT3, MAPK/Capsase-3, TRAIL Lectin-like ox-LDL-receptor-1, JAK-STAT, IRF3, Mst1-JNK, PI3K/AKT/mTOR, JNK, TLR4/MyD88/NF-κB, Dipeptidyl Peptidase-4, IRS1-GLUT4, ERK ½, Skp2-p27, PI3K/Akt, AP-1, Hedgehog, Adiponectin-AMPK. Akt/GSK-3-beta, RAF, ATM/ATR, MAPK/Erk, IKK/NF-κB, p38/ERK, TNF-α, Protein Tyrosine Phosphatase 1B, JNK/ERK VEGFR1 & VEGFR2, Ca 2+ levels, FOX, IRS/MAPK, NF-κB, IL-6 & IL-8, NF-κB/MAPK, COX-2, IL-8, and any combination thereof.

11. The method according to claim 6, wherein the STC(s) comprise(s) at least one selected from the group consisting of NF-κB, MAPK, COX-2, and JNK/ERK.

12. The method according to claim 11, wherein the STCs are NF-κB and MAPK, which STCs react synergistically with one another.

13. The method according to claim 11, wherein the STCs are COX-2 and JNK/ERK, which STCs react synergistically with one another.

14. A method of making the dosage form of claim 1, the method comprising admixing the selected phytonutrients and associating them together into or with the dosage form.

15. A supplement comprising multiple phytonutrients,
wherein the multiple phytonutrients are selected for each ability to maintain at least one signal transduction cascade in the subject, and wherein the selected multiple phytonutrients are present in an amount able to promote a subject's health through the maintenance of at least one signal transduction cascade in the subject.

16. A method of treating a female subject to reduce the symptoms of premenstrual syndrome (“PMS”), the method comprising:
administering the dosage form of claim 1 to the female subject so as to reduce the symptoms of PMS in the female subject.
